(19) United States
(12) Patent Application Publication
(10) Pub. No.: US 2010/0173954 A1
(43) Pub. Date: Jul. 8, 2010

(54) TREATMENT OF CANCERS HAVING RESISTANCE TO CHEMOTHERAPEUTIC AGENTS

(75) Inventors: Scott Wilhelm, Morristown, NJ (US); Richard W. Gedrich, Louisville, CO (US)

Correspondence Address: MILLEN, WHITE, ZELANO & BRANIGAN, P.C., 2200 CLARENDON BLVD., SUITE 1400, ARLINGTON, VA, 22201, US

(73) Assignee: BAYER HEALTHCARE LLC, Tarrytown, NY (US)

(21) Appl. No.: 12/523,697

(22) PCT Filed: Jan. 18, 2008
(86) PCT No.: PCT/US08/51405

§ 371 Date: Mar. 11, 2010

Related U.S. Application Data
(60) Provisional application No. 60/885,731, filed on Jan. 19, 2007.

(51) Int. Cl. A61K 31/44; A61P 35/00

(52) U.S. Cl. 514/350

(57) ABSTRACT

The present invention provides compositions and methods for treating cancer with DAST, 4{4-[3-(4-chloro-3-trifluoromethylphenyl)-ureido]-3-fluorophenoxy}-pyridine-2-carboxylic acid methylamide of the formula I, including all polymorphs, hydrates, pharmaceutically acceptable salts, metabolites, prodrugs, solvates or combinations thereof.

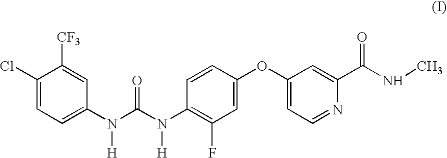

Any cancer can be treated, including cancers that have acquired resistance to another therapeutic agent, such as kinase inhibitors. DAST can also be used to treat cancers which have become refractory to other chemotherapeutic agents

### DESCRIPTION OF THE INVENTION

Cancer is a class of diseases characterized by two heritable properties: (1) uncontrolled cell division and (2) the ability of these cells to invade other tissues, either by direct growth into adjacent tissue (invasion) or by migration of cells to distant sites (metastasis). The hyper-proliferative properties initially give rise to a tumor or neoplasm. A tumor is considered a cancer when its cells acquire the ability to invade surrounding tissues, e.g., by breaking loose and entering the blood or lymph systems, or by forming secondary tumors at other sites in the body. The unregulated growth is caused by damaged DNA, resulting in mutations to vital genes that control cell division, the cell cycle, among other functions. One or more of these mutations, which can be inherited or acquired, can lead to uncontrolled cell division and cancer.

Cancers can be classified according to the tissue and cell type from which they arise. Cancers developing from epithelial cells are called carcinomas, and those from connective and muscle cells are called sarcomas. Additional cancers include those arising from hematopoietic cells (e.g., leukemia) and cancers of the nervous system.

In general, cancers appear to arise during a process in which an initial population of abnormal cells evolve into more aberrant cells through successive cycles of mutation and selection. More than 100 different genes have been identified which, when mutant, result in cancer. These so-called cancer-critical genes fall into two broad classes: oncogenes and tumor suppressor genes. Many cancer-critical genes play a role in the regulation of cell divisions, a highly complicated process involving multiple and parallel pathways. These include growth factors, cytokines, hormones, etc.

Cancer can cause many different symptoms, depending on the site and character of the malignancy and whether there is metastasis. A definitive diagnosis usually requires the microscopic examination of tissue obtained by biopsy. Once diagnosed, cancer is usually treated with surgery, chemotherapy and/or radiation.

If untreated, most cancers eventually cause death. Cancer is one of the leading causes of death in developed countries. It is estimated by the National Cancer Institute that approximately 9.8 million Americans were alive in January 2001 with a history of cancer. About 1,372,910 new cases of cancer were expected to be diagnosed in 2005, alone. In 2005, almost 600,000 Americans died of cancer, about 1 out of every 4 deaths. Many forms of cancer are associated with environmental factors, which may be avoidable. Smoking tobacco leads to more cancers than any other environmental factor.

Kinase inhibitors are being used successfully to treat cancers; however, some patients acquire a resistance to the drug's activity. In one embodiment, the present invention provides methods of treating a cancer in a subject in need thereof, comprising administering an effective amount of DAST to a subject having a cancer, wherein said cancer has acquired resistance to a kinase inhibitor. A kinase inhibitor is any drug or agent (e.g., anti-sense; small molecules; antibodies; etc) which blocks or reduces the activity of a kinase. This includes tyrosine kinases, serine-threonine kinases, receptor kinases, non-receptor kinases, etc. Generally, a “kinase activity” refers to the ability of a polypeptide to catalyze the transfer of a phosphate from one molecule to another.

There are a number of well-documented instances where cancers have acquired resistance to a kinase inhibitor which previously had successfully been used to treat the cancer. The term “acquired resistance” indicates that the cancer becomes resistant to the effects of the drug after being exposed to it for a certain period of time. For example, gastrointestinal stromal tumors (GIST), a mesenchymal tumor of the intestinal tract, and chronic myelogenous leukemia (CML) are treated with imatinib (STI571 or Gleevec), a tyrosine kinase inhibitor that inhibits the kinase activity of BCR-ABL, ABL, KIT, and PDGFR. It has shown been shown that, while patients may benefit from the treatment initially, many patients subsequently develop resistance to the agent. In some cases, this acquired resistance has been shown to result from a secondary mutation in the gene associated with the cancer. For example, most GIST patients have an activating mutation in either the KIT or PDGFRA gene. A study of GIST patients with acquired resistance to imatinib showed secondary mutations in the KIT kinase domain. See, e.g., Antonescu et al, Clin. Cancer Res., 11(11):41824190, 2005 and Heinrich et al., J. Clin. Oncology, 24(29), 4764-4774, 2006. A second site mutation in BCR-ABL is the predominant mechanism of imatinib resistance in CML. See, e.g., Gorre et al., Science, 293:876-880, 2001 who describe a mutation T315I associated with imatinib resistance. Acquired resistance has also been observed with other cancer drugs, including patients treated with EGFR-kinase inhibitors, such as gefitinib (Iressa) or erlotinib (Tarceva). See, e.g., Kobayshi et al., N. Engl. J. Med., 352:786-792, 2005. Pao et al. (PLoS Med., 2, e73, 2005) observed that patients with progressing lung tumors contained, in addition to a primary drug-sensitive mutation in EGFR, a secondary mutation in the kinase domain which led to drug-resistance.

Resistance mutations often occur in the kinase catalytic domain interfering or weakening the interaction with its inhibitor. Resistance mutations have been reported for number of kinases, including, BCR-ABL, KIT, PGF receptor, and EGF receptor. These secondary mutations often occur in the “gatekeeper” residue, the amino acid residue that “guards” the ATP-binding pocket and which also can comprise the site which interacts with the inhibitor. See, e.g., Noble et al., Science, 303: 1800-1805, 2004.

Nonetheless, the present invention relates to using DAST to treat a cancer which has acquired resistance to a kinase inhibitor, irrespective of the molecular mechanism responsible for it.

Examples of mutations which can lead to resistance and which can be treated in accordance with the presence invention include, e.g., KIT mutations, such as primary or secondary mutations in residues 654, 670, 816, 820, 822, 823, from residues about 650-654, about 670-674, from about 816-824, in the A-loop (activation), V654A, T670I, D816G, D816E, D820E, D820Y, N822K, and Y823D, etc. Such mutations, when secondary, can include any primary mutation, especially mutations in Exon 11, such as V559D, etc. Examples of mutations which lead to gefitinib and erlotinib resistance, include, e.g., mutations at residue 670, such as T790M (see also, Carter et al., Proc. Natl. Acad. Sci., 102:11011-11016, 2005).

Examples of kinase targets to which resistance can be acquired, included, but are not limited to, e.g., PDGFR-alpha, PDGFR-beta, EGFR, VEGFR, VEGFR1, VEGFR2, VEGFR3, HER-2, KIT, FLT3, c-MET, FGFR, FGFR1, FGFR3, c-FMS, RET, ABL, ALK, ARG, NTRK1m NTRK3, JAK2, ROS, etc. Associated cancers include, but are not limited to, CML (chronic myeloid leukemia), ALL (acute lymphoblastic leukemia), AML (acute myelogenous leukemia), T-ALL (T-Cell acute lymphoblastic leukemia), ALCL (acute lymphoblast cell leukemia), EMS (8p11 myeloproliferative syndrome), aCML (atypical chronic myelogenous leukemia), MM (multiple myeloma), T-lymphoma, MDS (myelodysplastic (syndrome), HES (hypereosinophilic syndrome), SM (systemic mastocytosis), and CMML (chronic myelomonocytic leukemia), IMT (inflammatory myofibroblastic tumor), NSCLC (non-small cell lung cancer), glioblastoma, SCCHN (squamous cell carcinoma of the head and neck), ovarian cancer, RCC (renal cell carcinoma), pancreatic cancer, colorectal cancer, breast cancer, lung cancer, GIST, seminoa, sarcomas, musculoskeletal tumors, gastric cancer, renal papillary carcinoma, malignant melanoma, PTC (papillary thyroid cancer), congenital fibrosarcoma, mesoblastic nephroma, secretory breast carcinoma, osteosarcoma, PAIS (pulmonary artery intimal sarcoma), DFSP (dermatofibrosarcoma protuberans), FMTC (familial medullary thyroid carcinoma), MEN-2B, radiation associated papillary thyroid cancer, astrocytoma, breast cancer, prostate cancer, renal cancer, etc. See, e.g., Krause et al., N. Engl. J., Med., 353:172-187, 2005. Diseases which can be treated in accordance with present invention include, e.g., diseases which are treated with imatinib, such as, but not limited to: Accelerated Phase Chronic Myelogenous Leukemia; Acute Erythroid Leukemia; Acute Lymphoblastic Leukemia; Acute Lymphoblastic Leukemia in Remission; Acute Lymphocytic Leukemia; Acute Monoblastic and Acute; Monocytic Leukemia; Acute Myelogenous Leukemia; Acute Myeloid Leukemia; Adenocarcinoma of the Prostate; Adenoid Cystic Carcinoma of the Head and Neck; Advanced Gastrointestinal Stromal Tumor; Agnogenic Myeloid; Metaplasia; Anaplastic Oligodendroglioma; Astrocytoma; B-Cell Adult Acute Lymphoblastic Leukemia; Blastic Phase Chronic Myelogenous Leukemia; Bone Metastases; Brain Tumor; Breast Cancer; Cancer; Central Nervous System Cancer; Childhood Acute Lymphoblastic Leukemia; Childhood Acute Lymphoblastic Leukemia in Remission; Childhood Central Nervous System Germ Cell Tumor; Childhood Chronic Myelogenous Leukemia; Childhood Soft Tissue Sarcoma; Chordoma; Chronic Eosinophilic Leukemia (CEL); Chronic Idiopathic Myelofibrosis; Chronic Myelogenous Leukemia; Chronic Myeloid Leukemia; Chronic Myelomonocytic Leukemia; Chronic Phase Chronic Myelogenous Leukemia; Colon Cancer; Colorectal Cancer; Dermatofibrosarcoma; Dermatofibrosarcoma Protuberans (DFSP); Desmoid Tumor; Eosinophilia; Epidemic Kaposi's Sarcoma; Essential Thrombocythemia; Ewing's Family of Tumors; Extensive Stage Small Cell Lung Cancer; Fallopian Tube Cancer; Familiar Hypereosinophilia; Fibrosarcoma; Gastric Adenocarcinoma; Gastrointestinal Neoplasm; Gastrointestinal Stromal Tumor; Glioblastoma; Glioma; Gliosarcoma; Grade I Meningioma; Grade II Meningioma; Grade III Meningioma; Hematopoietic and Lymphoid Cancer; High-Grade Childhood Cerebral Astrocytoma; Hypereosinophilic Syndrome; Idiopathic Pulmonary Fibrosis; L1 Adult Acute Lymphoblastic Leukemia; L2 Adult Acute Lymphoblastic Leukemia; Leukemia, Lymphocytic, Acute L2; Leukemia, Myeloid, Chronic; Leukemia, Myeloid, Chronic Phase; Liver Dysfunction and Neoplasm; Lung Disease; Lymphoid Blastic Phase of Chronic Myeloid Leukemia; Male Breast Cancer; Malignant Fibrous Histiocytoma; Mastocytosis; Meningeal Hemangiopericytoma; Meningioma; Meningioma; Meningioma; Metastatic Cancer; Metastatic Solid Tumors; Myelofibrosis; Myeloid Leukemia, Chronic; Myeloid Leukemia, Chronic Accelerated-Phase; Myeloid Leukemia, Chronic, Chronic-Phase; Myeloid Metaplasia; Myeloproliferative Disorder (MPD) with Eosinophilia; Neuroblastoma; Non-T, Non-B Childhood Acute Lymphoblastic Leukemia; Oligodendroglioma; Osteosarcoma; Ovarian Germ Cell Tumor; Ovarian Low Malignant Potential Tumor; Ovarian Neoplasms; Pancreatic Cancer; Pelvic Neoplasms; Peritoneal Cavity Cancer; Peritoneal Neoplasms; Philadelphia Chromosome Positive Chronic Myelogenous Leukemia; Philadelphia Positive Acute Lymphoblastic Leukemia; Philadelphia Positive Chronic Myeloid Leukemia in Myeloid Blast Crisis; Polycythemia Vera; Pulmonary Fibrosis; Recurrent Adult Brain Tumor; Recurrent Adult Soft Tissue Sarcoma; Recurrent Breast Cancer; Recurrent Colon Cancer; Recurrent Esophageal Cancer; Recurrent Gastric Cancer; Recurrent Glioblastoma Multiforme (GBM); Recurrent Kaposi's Sarcoma; Recurrent Melanoma; Recurrent Merkel Cell Carcinoma; Recurrent Ovarian Epithelial Cancer; Recurrent Pancreatic Cancer; Recurrent Prostate Cancer; Recurrent Rectal Cancer; Recurrent Salivary Gland Cancer; Recurrent Small Cell Lung Cancer; Recurrent Tumors of the Ewing's Family; Recurrent Uterine Sarcoma; Relapsing Chronic Myelogenous Leukemia; Rheumatoid Arthritis; Salivary Gland Adenoid Cystic Carcinoma; Sarcoma; Small Cell Lung Cancer; Stage II Melanoma; Stage II Merkel Cell Carcinoma; Stage III Adult Soft Tissue Sarcoma; Stage III Esophageal Cancer; Stage III Merkel Cell Carcinoma; Stage III Ovarian Epithelial Cancer; Stage III Pancreatic Cancer; Stage III Salivary Gland Cancer; Stage IIIB Breast Cancer; Stage IIIC Breast Cancer; Stage IV Adult Soft Tissue Sarcoma; Stage IV Breast Cancer; Stage IV Colon Cancer; Stage IV Esophageal Cancer; Stage IV Gastric Cancer; Stage IV Melanoma; Stage IV Ovarian Epithelial Cancer; Stage IV Prostate Cancer; Stage IV Rectal Cancer; Stage IV Salivary Gland Cancer; Stage IVA Pancreatic Cancer; Stage IVB Pancreatic Cancer; Systemic Mastocytosis; T-Cell Childhood Acute Lymphoblastic Leukemia; Testicular Cancer; Thyroid Cancer; Unresectable or Metastatic Malignant Gastrointestinal Stromal Tumor (GIST); Unspecified Adult Solid Tumor; Untreated Childhood Brain Stem Glioma; Uterine Carcinosarcoma, and Uterine Sarcoma.

Diseases which can be treated in accordance with present invention include, e.g., diseases which are treated with gefitinib, such as, but not limited to: Adenocarcinoma of the Colon; Adenocarcinoma of the Esophagus; Adenocarcinoma of the Lung; Adenocarcinoma of the Prostate; Adenocarcinoma of the Rectum; Advanced Adult Primary Liver Cancer; Advanced Non-Nasopharyngeal Head and Neck Carcinoma; Anaplastic Astrocytoma; Anaplastic Oligodendroglima; Anaplastic Thyroid Cancer; Bladder Cancer; Brain Tumor; Breast Cancer; Breast Cancer in Situ; Breast Neoplasms; Bronchoalveolar Cell Lung Cancer; Cancer of the Fallopian Tube; Carcinoma, Squamous Cell; Cervix Neoplasms; Colon Cancer; Colorectal Cancer; Epithelial Mesothelioma; Esophageal Cancer; Esophagogastric Cancer; Follicular Thyroid Cancer; Gastric Cancer; Gastrinoma; Gastrointestinal Carcinoid; Giant Cell Glioblastoma; Glioblastoma; Glioblastoma Multiforme; Head and Neck Cancer; Hepatocellular Carcinoma; Hypopharyngeal Cancer; Inoperable Locally Advanced Squamous Cell Carcinoma of Head and Neck; Insulinoma; Intraductal Breast Carcinoma; Islet Cell Carcinoma; Large Cell Lung Cancer; Laryngeal Cancer; Lip and Oral Cavity Cancer; Lip Cancer; Liver Cancer; Lung Adenocarcinoma With Bronchiolo-Alveolar Feature; Lung Cancer; Male Breast Cancer; Medullary Thyrod Cancer; Meningeal Tumors; Metastatic Colorectal Cancer; Metastatic Gastrointestinal Carcinoid Tumor; Metastatic Pancreatic Carcinoma; Mixed Gliomas; Myelogenous Leukemia, Acute; Nasopharyngeal Carcinoma; Neuroblastoma; Non-Metastatic (T2-T4, N0-N3, M0; Stages II and III) and Histologically-Confirmed Intestinal GC; Non-Metastatic Prostate Cancer; Nonresectable Adrenocortical Carcinoma; Non-Small Cell Lung Cancer; Nose Cancer; Oligodendroglial Tumors; Oral Cancer; Oropharyngeal Cancer; Osteosarcoma; Ovarian Cancer; Ovarian Neoplasms; Pancreatic Cancer; Papillary Thyroid Cancer; Peritoneal Carcinoma; Pharynx Cancer; Pneumonic-Type Adenocarcinoma (P-ADC); Primary Hepatocellular Carcinoma; Prostate Cancer; Rectal Cancer; Recurrent Adult Primary Liver Cancer; Recurrent Breast Cancer; Recurrent Colon Cancer; Recurrent Endometrial Cancer; Recurrent Esophageal Cancer; Recurrent Glioblastoma; Recurrent Rectal Cancer; Recurrent Skin Cancer; Refractory Germ Cell Tumors Expressing EGRF; Renal Cell Cancer; Rhabdomyosarcomas; Sarcomatous Mesothelioma; Skin Cancer; Soft Tissue Sarcoma; Squamous Cell Carcinoma of the Esophagus; Squamous Cell Carcinoma of the Head and Neck; Squamous Cell Carcinoma of the Skin; Squamous Cell Lung Cancer; Stage II Esophageal Cancer; Stage III Esophageal Cancer; Synovial Sarcoma; Thorax and Respiratory Cancer; Throat Cancer; Thyroid Cancer; Transitional Cell Cancer of the Renal Pelvis and Ureter; Transitional Cell Carcinoma of the Bladder; Tubal Carcinoma; Unspecified Childhood Solid Tumor; Untreated Childhood Brain Stem Glioma; Urethral Cancer.

Diseases which can be treated in accordance with present invention include, e.g., diseases which are treated with tarceva, such as, but not limited to: Adenocarcinoma; Adenocarcinoma of the Colon; Adenocarcinoma of the Esophagus; Adenocarcinoma of the Lung; Adenocarcinoma of the Pancreas; Adenocarcinoma of the Prostate; Adenocarcinoma of the Stomach; Adenosquamous Cell Lung Cancer; Adult Giant Cell Glioblastoma; Advanced Adult Primary Liver Cancer; Advanced NSCLC; Advanced Solid Tumors; Anaplastic Astrocytoma; Anaplastic Olligodendroglioma; Andrigen Deprivation Therapy; Bladder Cancer; Brenner Tumor; Bronchoalveolar Cell Lung Cancer; Childhood Brain Tumor; Childhood Cerebellar Astrocytoma; Childhood Cerebral Astrocytoma; Childhood Ependymoma; Childhood Malignant; Germ Cell Tumor; Childhood Oligodendroglioma; Colorectal Cancer; ECOG; Endometrial Adenocarcinoma; Endometrial Adenosquamous Cell; Esophageal Cancer; Extrahepatic Bile Duct Cancer; Fallopian Tube Cancer; Fallopian Tube Cancer; Female Reproductive Cancer; Gallbladder Cancer; Gastric Cancer; Gastrointestinal Cancer; Glioblastoma Multiforme; Gliosarcoma; Head and Neck Cancer; Head and Neck Neoplasms; High-Grade Childhood; Cerebral Astrocytoma; Hormone Sensitive Metastatic Breast Cancer; Hypopharyngeal Cancer; Kidney and Urinary Cancer; Laryngeal Cancer; Localized Unresectable Adult Primary liver Cancer; Low-Grade Childhood Cerebral Astrocytoma; Lung Adenocarcinoma With Bronchiolo-Alveolar Feature; Male Breast Cancer; Meningioma; Mesothelioma; Mixed Gliomas; Nasopharyngeal Cancer; Neoplasms; Neurofibrosarcoma; Non-Metastatic Prostate Cancer; Non-Small-Cell Lung; Oral Cavity Cancer; Oropharyngeal Cancer; Ovarian Cancer; Ovarian Epithelial Cancer; Ovarian Neoplasms; Pancreatic Cancer; Peritoneal Cavity Cancer; Pharynx Cancer; Pharynx Neoplasms; Pneumonic-Type Adenocarcinoma (P-ADC); Primary Hepatocellular Carcinoma; Primary Liver Cancer; Prostate Cancer; Prostate Cancer, Androgen Independent; Pulmonary Diseases; Recurrent Adult Brain Tumor; Recurrent Adult Primary Liver Cancer; Recurrent Breast Cancer; Recurrent Cervical Cancer; Recurrent Endometrial Cancer; Recurrent Esophageal Cancer; Recurrent Pancreatic Cancer; Recurrent Renal Cell Cancer; Relapsed/Refractory Non-Small-Cell Lung Cancer; Renal Cell Carcinoma; Rising Prostate Specific Antigen (PSA); Soft Tissue Sarcoma; Squamous Cell Carcinoma; Squamous Cell Carcinoma of the Esophagus; Squamous Cell Carcinoma of the Lip and Oral Cavity; Squamous Cell Carcinoma of the Oropharynx; Stage II Pancreatic Cancer; Stage III Pancreatic Cancer; Stage IIIA Non-Small Cell Lung Cancer; Stage IIIB or IV Non-Small Cell Lung Cancer; Stage IV Breast Cancer; Stage IV Colon Cancer; Stage IV Endometrial Cancer; Stage IV Rectal Cancer; Thorax and Respiratory Cancer; Transitional Cell Carcinoma of the Bladder; Tumors Metastatic to Brain; Unspecified Adult Solid Tumor; Upper Aerodigestive Tract Neoplasms.

Examples of tyrosine kinase inhibitors and other kinase inhibitors, include, but are not limited to, e.g., ABX-EGF, adaphostin, AEE788, AG 013736, AG 490, AG 825, AG 957, AG 1024, AG 1296, aloisine, aloisine A, alsterpaullone, aminogenistein, AMG 706, AMN 107, API-2, AP23573, apigenin, ARRY-142886 (AZD6244), arctigenin, AY-22989, AZD0530, AZD1152, AZD2171, bevacizumab, bisindolylmaleimide IX, BMS-354825, BMS-387032, BMS-599626, Bryostatin 1, CCI779, CEP-701, CEP-7055, cetuximab, 2C4, chelerythrine, CHIR-258, CI-1033, CPT-11, CP724714, CGP52421, CP-547-632, CT52923, CYC202, D816X, DMPQ, DRB, erlotinib (tarceva or OSI774), edelfosine, erbstatin analog, ET18OCH3, everolimus (RAD0001), fasudil, FK506, gefitinib (ZD1839), GO 6976, GW2974, GW572016, GW786034, imatinib mesylate (STI57 or Gleevec), H-7, H-8, H-89, HA-100, HA-1004, HA-1077, HA-1100, hydroxyfasudil, Isis 3521, indirubin-3′-oxime, 5-Iodotubercidin, kenpaullone, KN-62, KY12420, lapatinib ditosylate (GSK572016), LFM-A13, limofosine, luteolin, LY294002, LY294002, LY333531, LY379196, mallotoxin, midostaurin, ML-9, MLN518, NSC-154020, NSC-226080, NSC-664704, NSC-680410, NU6102, olomoucine, oxindole I, PD 0173074, PD 0325901, PD 153035, PD 98059, PD 169316, PD 184352, phloridzin, Perifosine, PKC412, piceatannol, picropodophyllin, PP1, PP2, purvalanol A, PTK787 (ZK 2222584; vatalanib), quercetin, RAPA, rapamune, rapamycin, R0 318220, R0 320432, roscovitine, rottlerin, SB202190, SB203580, sirolimus, SL327, SMS-354825, SP600125, staurosporine, STI-571, SU101, SU1498, SU4312, SU6656, SU5402, SU5416, SU6668, SU11248, sunitinib (sutent), syk inhibitor, TBB, TCN, Triciribine, Tyrphostin AG 490, Tyrphostin AG 825, Tyrphostin AG 957, Tyrphostin AG 1024, trastuzumab (herceptin), wortmannin, XL647, XL999, Y-27632, U0126, UCN-01, VX-680, ZD6474, ZM 252868, and analogs and derivatives thereof, etc.

Specific examples of tyrosine kinase inhibitors include, e.g., AEE788, AMG 706, AMN107, ARRY-142886 (AZD6244), AZD2171, AZD0530, bevacizumab, BMS-354825, BMS-599626, CCI779, CEP-7055, cetuximab, CHIR-258, CI-1033, CP-724714, CP-547-632, erlotinib (tarceva or OSI774), gefitinib (Iressa), GW572016, GW786034, imatinib mesylate (STI57 or Gleevec), lapatinib ditosylate (GSK572016), PD 0173074, PD 0325901, PKC412, PTK787, rapamycin, sunitinib (sutent), SU5416, SU11248, SU6668, trastuzumab, XL647, ZD6474, and analogs and derivatives thereof.

Further examples of tyrosine kinase inhibitors, include, e.g., 17-DMAG; 17-AAG; AG 9; AG 10; AG 1; AG 18; AG 30; AG 43; AG 82; AG 99; AG 112; AG 126; AG 183; AG 213; AG 370: AG 490; AG 494; AG 527; AG 537; AG 538; AG 555; AG 556; AG 592; AG 825; AG 835; AG 879; AG 957; AG 957; AG 1024; AG 1288; AG 1295; AG 1296; AG 1387; AG 1433; AG 1478; AGL 2043; AGL 2263; Aminogenistein; BPDQ; BPIQ-I; BPIQ-II; 4-[(3′-Bromo-4′-hydroxyphenyl)amino]-6,7-dimethoxyquinazoline (WHI-P154); 4-[(3-Bromophenyl)amino]-6,7-diethoxyquinazoline; Butein; (5-Butanoate-1H-2-indolyl)(1H-2-indolyl)-methanone; 4-[(4′-Chloro-2′-fluoro)phenylamino]-6,7-dimethoxyquinazoline; N-(4-Chlorophenyl)-2-[(pyridin-4-ylmethyl)amino]benzamide; CL-387785; Cucurbitacin I, Cucumis sativus L.; Curcumin, Curcuma longa L.; Daidzein; Damnacanthal; Daphnetin; 5′-Deoxy-5′-methylthioadenosine; 4-(3′,5′-Dibromo-4-hydroxyphenyl)amino-6,7-dimethoxyquinazoline (WHI-P97); (Z)-5-Bromo-3-(4,5,6,7-tetrahydro-1H-indol-2-ylmethylene)-1,3-dihydroindol-2-one; 2-(1,1-Dimethylethyl)-9-fluoro-3,6-dihydro-7H-benz[h]-imidaz[4,5-f]isoquinolin-7-one; 4-(6,7-Dimethoxy-4-quinazolinyl)-N-(4-phenoxyphenyl)-1-piperazinecarboxamide; 3-[(2,4-Dimethylpyrrol-5-yl)methylidene]-indolin-2-one (SU5416); (Z)-3-[(2,4-Dimethyl-3-(ethoxycarbonyl)pyrrol-5-yl)methylidenyl]indolin-2-one; DMBI; Emodin; Erbstatin Analog; Geldanamycin (Streptomyces hygroscopicus); Genistein; Genistin; GTP-14564;

Other examples of tyrosine kinase inhibitors, include, but are not limited to, Herbimycin A (Streptomyces sp.); 1,2,3,4,5,6-Hexabromocyclohexane; HNMPA-(AM)3; (5-Hydroxy-1H-2-indolyl) (1H-2-indolyl)-methanone; 4-(4′-Hydroxyphenyl)amino-6,7-dimethoxyquinazoline (WHI-P131); IGF-1R Inhibitor, PPP; I-OMe-AG 538; (Z,E)-3-(Imidazol-4-ylmethylene)indolin-2-one; [2-(1H-2-Indolylcarbonyl)-1H-5-indolyl]butanoate; Indirubin Derivative E804; K-252a, Nocardiopsis sp.; Lavendustin A; Lavendustin B; LFM-A11; LFM-A12; LFM-A13; MAZ51; 3-(1-Methyl-1H-indol-3-yl-methylene)-2-oxo-2,3-dihydro-1H-indole-5-sulfonamide; 2-Naphthyl-(N-isopropyl,N-benzyl)-b-aminoethylketone, HCl; 2-Naphthylvinyl Ketone; Oxindole I; PD 153035; PD 156273; PD 158780; PD 168393; PD 174265; Piceatannol; PP1 Analog; PP1 Analog II (1NM-PP1); PP2; PP3; Quercetin; Radicicol (Diheterospora chlamydosporia); RG-13022; 4-(4′-Phenoxyanilino)-6,7-dimethoxyquinazoline; p60v-src 137-157 Inhibitor Peptide (VAPSDSIQAEEWYFGKITRRE); ST638; SU11652; SU1498; SU4984; SU5402; SU5614; SU6656; (−)-Terreic Acid, Synthetic; 2′-Thioadenosine; Tyrene CR4; 3-(3-Thienyl)-6-(4-methoxyphenyl)pyrazolo[1,5-a]pyrimidine; ZM323881; ZM 39923; and ZM 449829.

As indicated above, the present invention provides methods of treating cancers which have acquired resistance to a kinase inhibitor comprising, e.g., comprising administering to a subject in need thereof an effective amount of DAST, wherein the cancer is treated.

The phrase “effective amount” indicates the amount of DAST which is effective to treat any symptom or aspect of the cancer. Effective amounts can be determined routinely. Further guidance on dosages and administration regimens is provided below.

The term “treating” is used conventionally, e.g., the management or care of a subject for the purpose of combating, alleviating, reducing, relieving, improving, etc., one or more of the symptoms associated with a cancer, including all cancers mentioned herein. Administering effective amounts of DAST can treat one or more aspects of the cancer disease, including, but not limited to, causing tumor regression; causing cell death; causing apoptosis; causing necrosis; inhibiting cell proliferation; inhibiting tumor growth; inhibiting tumor metastasis; inhibiting tumor migration; inhibiting tumor invasion; reducing disease progression; stabilizing the disease; reducing or inhibiting angiogenesis; prolonging patient survival; enhancing patient's quality of life; reducing adverse symptoms associated with cancer; and reducing the frequency, severity, intensity, and/or duration of any of the aforementioned aspects.

Any cancer can be treated in accordance of the present invention, irrespective of the type or cause of the cancer, and irrespective of the genetic lesions associated with it (see, e.g., Atlas of Genetics and Cytogenetics in Oncology and Haematology on the worldwide web at infobiogen.fr/services/chromcancer/for an atlas of genes involved in cancer). In addition to treating cancer, pre-cancerous cells, tumors, neoplasms, and non-malignant tumors can also be treated.

Cancers which can be treated include, e.g., cancers which are primary; which arise from a primary tumor at a secondary metastatic site; which have been treated by surgery (e.g., entirely removed, surgical resection, etc); which have been treated by chemotherapy, radiation, radiofrequency ablation, and/or any other adjunct to drug therapy; which have acquired drug-resistance; which are refractory to a chemotherapeutic agent.

Any subject can be in accordance with the present invention, including, e.g., mammals, such as dogs, cats, horses, rats, mice, monkeys, and humans.

In addition to kinase inhibitors, the present invention also relates to treating a cancer which has acquired resistance to any agent targeted at the RAS-RAF-MEK-ERK pathway. Agents include any compound which is an inhibitor of any component of the aforementioned pathway.

The present invention also provides methods of treating a cancer in a subject in need thereof, comprising administering an effective amount of DAST to said subject having a cancer, wherein said cancer is refractory to a chemotherapeutic agent. The term “refractory” means, e.g., that the cancer (including a tumor and/or any metastasis thereof), upon treatment with at least one chemotherapeutic shows no or only weak anti-cancer (e.g., anti-proliferative response) (such as, no or only weak inhibition of tumor growth) after the treatment with such an agent. Thus, after a patient has been treated with a chemotherapeutic agent with success, but subsequent treatments show no or little affect, the cancer can be described as being refractory to the agent. Examples of chemotherapeutic agents include, but are not limited to, e.g., alkylating agents (e.g., cyclophosphamide, ifosfamide, melphalan, chlorambucil, aziridines, epoxides, alkyl sulfonates), cisplatin and its analogues (e.g., carboplatin, oxaliplatin), antimetabolitites (e.g., methotrexate, 5-fluorouracil, capecitabine, cytarabine, gemcitabine, fludarabine), toposiomerase interactive agents (e.g., camptothecin, irinotecan, topotecan, etoposide, teniposide, doxorubicin, daunorubicin), antimicrotubule agents (e.g., vinca alkaloids, such as vincristine, vinblastine, and vinorelbine; taxanes, such as paclitaxel and docetaxel), interferons, interleukin-2, histone deacetylase inhibitors, monoclonal antibodies, estrogen modulators (e.g., tamoxifen, toremifene, raloxifene), megestrol, aromatase inhibitors (e.g., letrozole, anastrozole, exemestane, octreotide), octreotide, anti-androgens (e.g., flutamide, casodex), etc. See, e.g. Cancer: Principles and Practice of Oncology, 7th Edition, Devita et al, Lippincott Williams & Wilkins, 2005, Chapters 15, 16, 17, and 63.

The term “DAST” as used herein refers to the compound: 4{4-[3-(4-chloro-3-trifluoromethylphenyl)-ureido]-3-fluorophenoxy}-pyridine-2-carboxylic acid methylamide of the formula I below including all polymorphs, hydrates, solvates, pharmaceutically acceptable salts or combinations thereof. Also included are the metabolites of 4{4-[3-(4-chloro-3-trifluoromethylphenyl)-ureido]-3-fluorophenoxy}-pyridine-2-carboxylic acid methylamide and prodrugs of 4{4-[3-(4-chloro-3-trifluoromethylphenyl)-ureido]-3-fluorophenoxy}-pyridine-2-carboxylic acid methylamide prepared by conventional techniques.

Suitable pharmaceutically acceptable salts are well known to those skilled in the art and include salts of inorganic and organic acids, such as hydrochloric acid, hydrobromic acid, sulfuric acid, phosphoric acid, methanesulphonic acid, trifluoromethanesulfonic acid, benzenesulfonic acid, 1-naphthalenesulfonic acid, 2-naphthalenesulfonic acid, acetic acid, trifluoroacetic acid, malic acid, tartaric acid, citric acid, lactic acid, oxalic acid, succinic acid, fumaric acid, maleic acid, benzoic acid, salicylic acid, phenylacetic acid, and mandelic acid. In addition, pharmaceutically acceptable salts include salts of inorganic bases, such as salts containing alkaline cations (e.g., Li⁺ Na⁺ or K⁺), alkaline earth cations (e.g., Mg⁺², Ca⁺² or Ba⁺²), the ammonium cation, as well as acid salts of organic bases, including aliphatic and aromatic substituted ammonium, and quaternary ammonium cations, such as those arising from protonation or peralkylation of triethylamine, N,N-diethylamine, N,N-dicyclohexylamine, lysine, pyridine, N,N-dimethylaminopyridine (DMAP), 1,4-diazabiclo[2.2.2]octane (DABCO), 1,5-diazabicyclo[4.3.0]non-5-ene (DBN) and 1,8-diazabicyclo[5.4.0]undec-7-ene (DBU).

Solvates for the purposes of the invention are those forms of the compound where solvent molecules form a complex in the solid state and include, but are not limited to for example ethanol and methanol. Hydrates are a specific form of solvates, where the solvent molecule is water.

The metabolites of DAST include oxidized derivatives wherein one or more of the urea nitrogens shown in of Formula I are substituted with a hydroxyl group. The metabolites of DAST also include analogs where the methylamide group shown in Formula I is hydroxylated then de-methylated by metabolic degradation. The metabolites of DAST further include oxidized derivatives where the pyridine nitrogen atom shown in of Formula I is in the N-oxide form (e.g. carries a hydroxy substituent) leading to those structures referred to in the art as 1-oxo-pyridine and 1-hydroxy-pyridine.

DAST can be further modified with labile functional groups that are cleaved after in vivo administration to furnish the parent active agent and the pharmacologically inactive derivatizing (functional) group. These derivatives, commonly referred to as prodrugs, can be used, for example, to alter the physicochemical properties of the active agent, to target the active agent to a specific tissue, to reduce undesirable side effects and/or to alter the pharmacokinetic and pharmacodynamic properties of the active agent (e.g., solubility, absorption, biostability and release time, see “Pharmaceutical Dosage Form and Drug Delivery Systems” (Sixth Edition), edited by Ansel et al., published by Williams & Wilkins, pages 27-29, (1995) which is hereby incorporated by reference),

Suitable including N-dealkylation, O-dealkylation, aliphatic hydroxylation, aromatic hydroxylation, N-oxidation, S-oxidation, deamination, hydrolysis reactions, glucuronidation, sulfation and acetylation (see Goodman and Gilman's The Pharmacological Basis of Therapeutics (Ninth Edition), editor Molinoff et al., pub. by McGraw-Hill, pages 11-13, (1996), which is hereby incorporated by reference).

Suitable prodrugs of DAST include, e.g., well-tolerated, pharmaceutically acceptable esters such as alkyl esters including methyl, ethyl, propyl, isopropyl, butyl, isobutyl or pentyl esters. Additional esters such as phenyl-C₁-C₅ alkyl esters may be used, although methyl ester is preferred.

Methods for synthesizing prodrugs are described in the following reviews on the subject, which are incorporated herein by reference for their description of these methods:
Higuchi, T.; Stella, V. eds. Prodrugs as Novel Drug Delivery Systems. ACS Symposium Series. American Chemical Society: Washington, D.C. (1975).
Roche, E. B. Design of Biopharmaceutical Properties through Prodrugs and Analogs. American Pharmaceutical Association: Washington, D.C. (1977).
Sinkula, A. A.; Yalkowsky, S. H. J Pharm Sci. 1975, 64, 181-210.
Stella, V. J.; Charman, W. N. Naringrekar, V. H. Drugs 1985, 29, 455-473.
Bundgaard, H., ed. Design of Prodrugs. Elsevier: N.Y. (1985).
Stella, V. J.; Himmelstein, K. J. J. Med. Chem. 1980, 23, 1275-1282.
Han, H-K; Amidon, G. L. AAPS Pharmsci 2000, 2, 1-11.
Denny, W. A. Eur. J. Med. Chem. 2001, 36, 577-595.
Wermuth, C. G. in Wermuth, C. G. ed. The Practice of Medicinal Chemistry Academic Press: San Diego (1996), 697-715.
Balant, L. P.; Doelker, E. in Wolff, M. E. ed. Burgers Medicinal Chemistry And Drug Discovery John Wiley & Sons: New York (1997), 949-982.

Formula I is as follows:

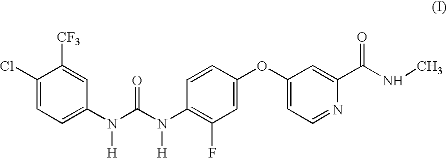

Examples of the preparation of DAST, salts thereof and pharmaceutical compositions thereof follow.

### Preparation of the Intermediate: 4-amino-3-fluorophenol

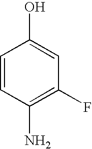

To a dry flask purged with Argon was added 10% Pd/C (80 mg) followed by 3-fluoro-4-nitrophenol (1.2 g, 7.64 mmol) as a solution in ethyl acetate (40 mL). The mixture was stirred under an H₂ atmosphere for 4 h. The mixture was filtered through a pad of Celite and the solvent was evaporated under reduced pressure to afford the desired product as a tan solid (940 mg, 7.39 mmol; 97% yield); ¹H-NMR (DMSO-d₆) 4.38 (s, 2H), 6.29-6.35 (m, 1H), 6.41 (dd, J=2.5, 12.7, 1H), 6.52-6.62 (m, 1H), 8.76 (s, 1H).

### Preparation of the Starting Material 1: 4-(4-amino-3-fluorophenoxy)pyridine-2-carboxylic acid methylamide

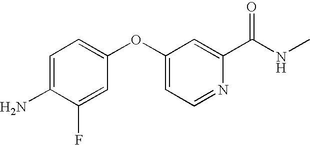

A solution of intermediate 4-amino-3-fluorophenol, (500 mg, 3.9 mmol) in N,N-dimethylacetamide (6 mL) cooled to 0° C. was treated with potassium tert-butoxide (441 mg, 3.9 mmol), and the brown solution was allowed to stir at 0° C. for 25 min. To the mixture was added 4-chloro-N-methyl-2-pyridinecarboxamide, (516 mg, 3.0 mmol) as a solution in dimethylacetamide (4 mL). The reaction was heated at 100° C. for 16 h. The mixture was cooled to room temperature, quenched with H₂O (20 mL), and extracted with ethylacetate (4×40 mL). The combined organics were washed with H₂O (2×30 mL), dried (MgSO₄), and evaporated to afford a red-brown oil. ¹H-NMR indicated the presence of residual dimethylacetamide, thus the oil was taken up in diethylether (50 mL) and was further washed with brine (5×30 mL). The organic layer was dried (MgSO₄) and concentrated to give 950 mg of the desired product, starting material 1, as a red-brown solid, which was used in the next step without purification.

### EXAMPLE 1

### Preparation of DAST: 4{4-[3-(4-chloro-3-trifluoromethylphenyl)-ureido]-3-fluorophenoxy}-pyridine-2-carboxylic acid methylamide

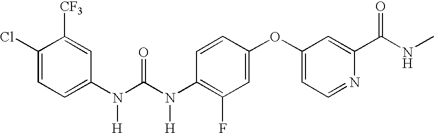

To a solution of 4-(4-amino-3-fluorophenoxy)pyridine-2-carboxylic acid methylamide (starting material 1, 177 mg, 0.68 mmol) in toluene (3 mL) was added 4-chloro-3-(trifluoromethyl)phenyl isocyanate (150 mg, 0.68 mmol). The mixture was stirred at room temperature for 72 h. The reaction was concentrated under reduced pressure and the residue was triturated with diethylether. The resulting solid was collected by filtration and dried in vacuo for 4 h to afford the title compound (155 mg, 0.32 mmol; 47% yield); ¹H-NMR (DMSO-d₆) 2.78 (d, J=4.9, 3H), 7.03-7.08 (m, 1H), 7.16 (dd, J=2.6, 5.6, 1H), 7.32 (dd, J=2.7, 11.6, 1H), 7.39 (d, J=2.5, 1H), 7.60 (s, 2H), 8.07-8.18 (m, 2H), 8.50 (d, J=5.7, 1H), 8.72 (s, 1H), 8.74-8.80 (m, 1H), 9.50 (s, 1H); MS (HPLC/ES) 483.06 m/z=(M+1).

### EXAMPLE 2

### Preparation of the Salt: 4{4-[3-(4-chloro-3-trifluoromethylphenyl)-ureido]-3-fluorophenoxy}-pyridine-2-carboxylic acid methylamide hydrochloride

The compound of Example 1 as a free base (2.0 g) was dissolved in anhydrous tetrahydrofuran (15 mL) and a 4M HCl/dioxane was added (excess). The solution was then concentrated in vacuo to afford 2.32 grams of off-white solids. The crude salt was dissolved in hot ethanol (125 mL), activated carbon was added and the mixture heated at reflux for 15 minutes. The hot suspension was filtered through a pad of Celite 521 and allowed to cool to room temperature. The flask was placed in a freezer overnight. The crystalline solids were collected by suction filtration, washed with ethanol, then hexane and air-dried. The mother liquors were concentrated down and crystallization (in freezer) allowed taking place overnight. A second crop of solids was collected and combined with the first crop. The colorless salt was dried in a vacuum oven at 60° C. over two days. Yield of hydrochloride salt obtained 1.72 g (79%).

Melting Point: 215° C.

Elemental Analysis:

| | Calcd. | Found |
|---|---|---|
| C | 48.57 | 48.68 |
| H | 3.11 | 2.76 |
| N | 10.79 | 10.60 |
| Cl | 13.65 | 13.63 |
| F | 14.63 | 14.88 |

### EXAMPLE 3

### Preparation of the Salt: 4{4-[3-(4-chloro-3-trifluoromethylphenyl)-ureido]-3-fluorophenoxy}-Pyridine-2-carboxylic acid methylamide mesylate

The compound of Example 1 as a free base (2.25 g) was dissolved in ethanol (100 mL) and a stock solution of methanesulfonic acid (excess) was added. The solution was then concentrated in vacuo to afford a yellow oil. Ethanol was added and concentration repeated, affording 2.41 g of off-white solids. The crude salt was dissolved in hot ethanol (˜125 mL) and then cooled slowly to crystallize. After reaching room temperature, the flask was placed in a freezer overnight. The colorless crystalline material was collected by suction filtration; the filter cake was washed with ethanol, then hexane and air-dried, to afford 2.05 g of material, which was dried in a vacuum oven at 60° C. overnight.

Melting Point: 231° C.

Elemental Analysis:

| | Calcd. | Found |
|---|---|---|
| C | 45.64 | 45.34 |
| H | 3.31 | 3.08 |
| N | 9.68 | 9.44 |
| Cl | 6.12 | 6.08 |
| F | 13.13 | 13.42 |
| S | 5.54 | 5.59 |

### EXAMPLE 4

### Preparation of the Salt: 4{4-[3-(4-chloro-3-trifluoromethylphenyl)-ureido]-3-fluorophenoxy}-Pyridine-2-carboxylic acid methylamide phenylsulfonate

The compound of Example 1 as a free base (2.25 g) was suspended in ethanol (50 mL) and benzensulfonic acid (0.737 g) in ethanol (50 mL) was added. The mixture was heated with vigorous stirring. All solid material dissolved to give a reddish solution. The solution was allowed to cool to room temperature and the flask scratched. Crystal formation was slow, some seeds were found, added to solution and placed in freezer overnight. Grayish-tan solids had formed in the flask; the material was broken up & collected by suction filtration. The solids were washed with ethanol, then hexane and air-dried. Weighed product: 2.05 g, 69% yield.

Melting Point: 213° C.

Elemental Analysis:

| | Calcd. | Found |
|---|---|---|
| C | 50.59 | 50.24 |
| H | 3.30 | 3.50 |
| N | 8.74 | 8.54 |
| F | 11.86 | 11.79 |
| Cl | 5.53 | 5.63 |
| S | 5.00 | 5.16 |

### EXAMPLE 5

### Preparation of a 1+4 Solid Dispersion of 4{4-[3-(4-chloro-3-trifluoromethylphenyl)-ureido]-3-fluorophenoxy}pyridine-2-carboxylic acid methyl amide with polyvinylpyrrolidone

In an uncapped vial, one part of the compound of Example 1 as a free base was mixed with four parts polyvinylpyrrolidone (PVP-25/Kollidon® 25), and dissolved in a sufficient amount of a 1:1 mixture of acetone and ethanol, until all powders are in solution. The uncapped vial was placed into a vacuum oven set at 40° C., and let dry for at least 24-48 hours.

### EXAMPLE 6

### Preparation of a 1+3 Solid Dispersion of 4{4-[3-(4-chloro-3-trifluoromethylphenyl)-ureido]-3-fluorophenoxy}-pyridine-2-carboxylic acid methyl amide with polyvinylpyrrolidone

One part of the compound of Formula I as base and three parts of polyvinylpyrrolidone (PVP 25/Kollidon® 25) were dissolved in 30 parts of a 80:20 acetone/ethanol mixture (w/w). Using a rotary vacuum evaporator the solvent was removed at 70° C. The dry residue was removed from the evaporation flask and sieved (630 μm).

### EXAMPLE 7

### Preparation of a 1+7 Solid Dispersion of 4{4-[3-(4-chloro-3-trifluoromethylphenyl)-ureido]-3-fluorophenoxy}-pyridine-2-carboxylic acid methyl amide with polyvinylpyrrolidone

One part of the compound of Formula I as base and seven parts PVP 25 were dissolved in 30 parts of a 80:20 acetone/ethanol mixture (w/w). Using a rotary vacuum evaporator the solvent was removed at 70° C. The dry residue was removed from the evaporation flask and sieved (630 μm).

### EXAMPLE 8

### Solid Dispersion of 4{4-[3-(4-chloro-3-trifluoromethylphenyl)-ureido]-3-fluorophenoxy}-pyridine-2-carboxylic acid methyl amide with hydroxypropyl cellulose (HPC) Prepared by Melt Extrusion

Two parts of the compound of Formula I as base were mixed with one part of Maltitol and seven parts of HPC-M. The mixture was extruded using a lab twin screw extruder at a temperature of 160-200° C. The extruded material was cut and subsequently milled using an impact lab mill. The resulting powder can be used as it is or it can be further formulated for example to sachet, capsule or tablet formulations.

### EXAMPLE 9

### Solid dispersion of 4{4-[3-(4-chloro-3-trifluoromethylphenyl)-ureido]-3-fluorophenoxy}-pyridine-2-carboxylic acid methyl amide with PVP and croscarmellose sodium

A solution of 0.4 kg of the of the compound of Formula I as base and 1.2 kg of PVP 25 in a mixture of 6.4 kg acetone and 1.6 kg ethanol was prepared. Using a fluidized bed vacuum granulator this solution was sprayed onto a powder bed of 1.6 kg croscarmellose sodium at a temperature of 60-70° C. After drying the product was sieved (1 mm). The granulate can be used as it is or it can be further formulated for example to sachet, capsule or tablet formulations.

### EXAMPLE 10

### Solid dispersion of 4{4-[3-(4-chloro-3-trifluoromethylphenyl)-ureido]-3-fluorophenoxy}-pyridine-2-carboxylic acid methyl amide with PVP and sodium starch glycolate

This material was prepared in a similar way as described in Example 9, except that the solution is sprayed onto a powder bed of 1.6 kg sodium starch gycolate Type A (Explotab®)

### EXAMPLE 11

### Solid dispersion of 4{4-[3-(4-chloro-3-trifluoromethylphenyl)-ureido]-3-fluorophenoxy}-pyridine-2-carboxylic acid methyl amide with PVP and croscarmellose sodium

A solution of 0.4 kg of the of the compound of Formula I as base and 1.6 kg of PVP 25 in a mixture of 6.4 kg acetone and 1.6 kg ethanol was prepared. Using a fluidized bed vacuum granulator this solution was sprayed onto a powder bed of 2 kg croscarmellose sodium at a temperature of 60-70° C. After drying the product was sieved (1 mm). The granulate can be used as it is or it can be further formulated for example to sachet, capsule or tablet formulations.

### EXAMPLE 12

### Solid dispersion of 4{4-[3-(4-chloro-3-trifluoromethylphenyl)-ureido]-3-fluorophenoxy}-pyridine-2-carboxylic acid methyl amide with PVP, croscarmellose sodium and microcrystalline cellulose

This material was prepared in a similar way as described in Example 11, except that the solution was sprayed onto a powder bed consisting of 1 kg croscarmellose sodium and 1 kg microcrystalline cellulose.

### EXAMPLE 13

### Solid dispersion of 4{4-[3-(4-chloro-3-trifluoromethylphenyl)-ureido]-3-fluorophenoxy}-pyridine-2-carboxylic acid methyl amide with HPC-SL and croscarmellose sodium

A solution of 0.4 kg of the of the compound of Formula I as base and 1.6 kg of HPC-SL in 20 kg acetone was prepared. Using a fluidized bed vacuum granulator this solution was sprayed onto a powder bed of 2 kg croscarmellose sodium at a temperature of 40-60° C. After drying the product was sieved (1 mm). The granulate can be used as it is or it can be further formulated for example to sachet, capsule or tablet formulations.

### EXAMPLE 14

### Solid dispersion of 4{4-[3-(4-chloro-3-trifluoromethylphenyl)-ureido]-3-fluorophenoxy}-pyridine-2-carboxylic acid methyl amide with HPC-L and croscarmellose sodium

A solution of 0.4 kg of the of the compound of Formula I as base and 1.6 kg of HPC-L in 28 kg acetone was prepared. Using a fluidized bed vacuum granulator this solution was sprayed onto a powder bed of 2 kg croscarmellose sodium at a temperature of 40-60° C. After drying the product was sieved (1 mm). The granulate can be used as it is or it can be further formulated for example to sachet, capsule or tablet formulations.

### EXAMPLE 15

### Tablets containing a solid dispersion of 4{4-[3-(4-chloro-3-trifluoromethylphenyl)-ureido]-3-fluorophenoxy}pyridine-2-carboxylic acid methyl amide

The granulate of Example 11 was roller compacted and screened 3 and 1 mm. Subsequently the compacted granulate was blended with 0.54 kg croscarmellose sodium, 24 g colloidal anhydrous silica and 36 g magnesium stearate. This ready-to-press blend was compressed on a rotary tablet press to tablets containing 20, 50 an 100 mg of the compound of Formula I. The tablets may be film-coated for light protection.

### EXAMPLE 16

### Tablets containing a solid dispersion of 4{4-[3-(4-chloro-3-trifluoromethylphenyl)-ureido]-3-fluorophenoxy}-pyridine-2-carboxylic acid methyl amide

The granulate of Example 12 was roller compacted and screened 3 and 1 mm. Subsequently the compacted granulate was blended with 0.54 kg croscarmellose sodium, 24 g colloidal anhydrous silica and 36 g magnesium stearate. This ready-to-press blend was compressed on a rotary tablet press to tablets containing 20, 50 an 100 mg of the compound of Formula I. The tablets may be film-coated for light protection.

### EXAMPLE 17

### Tablets containing a solid dispersion of 4{4-[3-(4-chloro-3-trifluoromethylphenyl)-ureido]-3-fluorophenoxy}-pyridine-2-carboxylic acid methyl amide

A solution of 0.4 kg of the of the compound of Formula I as base and 1.2 kg of PVP 25 in a mixture of 6.4 kg acetone and 1.6 kg ethanol was prepared. Using a fluidized bed vacuum granulator this solution was sprayed onto a powder bed consisting of 0.8 kg croscarmellose sodium and 0.8 kg microcrystalline cellulose at a temperature of 60-70° C. After drying the product is sieved (1 mm). The granulate is roller compacted and screened 3 and 1 mm. Subsequently the compacted granulate was blended with 1.34 kg croscarmellose sodium, 24 g colloidal anhydrous silica and 36 g magnesium stearate. This ready-to-press blend is compressed on a rotary tablet press to tablets containing 20, 50 an 100 mg of the compound of Formula I. The tablets may be film-coated for light protection.

The specific dose level and frequency of dosage may vary, depending upon a variety of factors, including the activity of the active agent, its metabolic stability and length of action, rate of excretion, mode and time of administration, the age, body weight, health condition, gender, diet, baseline hematologic and biologic parameters (e.g., WBCs, granulocytes, platelets, hemoglobin, creatinine, bilirubin, albumin, etc.), etc., of the subject, and the severity, intensity, stage of the cancer, primary site of cancer, size of cancer lesion, presence or extent of metastases, surgical status, disease progression (i.e., aggressive), etc. of the disease.

DAST can be administered in any form by any effective route, including, e.g., oral, parenteral, enteral, intraperitoneal, topical, transdermal (e.g., using any standard patch), ophthalmic, nasally, local, non-oral, such as aerosal, spray, inhalation, subcutaneous, intravenous, intramuscular, buccal, sublingual, rectal, vaginal, intra-arterial, intrathecal, intratumoral, etc. DAST can be administered directly to the site of a tumor, either pre- or post-operatively. It can be administered alone, or in combination with any ingredient(s), active or inactive.

DAST can be administered by the oral route using the pharmaceutical composition of the present invention. Dosages will generally range, based on body weight, from about 0.01 mg/kg to about 50 mg/kg; from about 1 mg/kg to about 40 mg/kg; from about 5 mg/kg to about 30 mg/kg; from about 10 to about 25 mg/kg; about 10 mg/kg; about 20 mg/kg; about 25 mg/kg; about 30 mg/kg; etc.

Any suitable dosing interval can be used in accordance with the present invention. For example, DAST can be administered once, twice (BID), three, four, etc., times a day. For example, about 100, about 200, about 400 mg, about 500 mg, about 600 mg, or about 800 mg can be administered one, twice, or three times daily.

DAST can be administered at any suitable time. For example, it can be administered routinely as other chemotherapeutic agents; it can be administered as a bolus prior to a surgical intervention; prior to or after radiation, radiofrequency ablation and other energy treatments; post-operatively; pre-operatively; etc.

DAST can be further combined with any other suitable additive or pharmaceutically acceptable carrier. Such additives include any of those used conventionally, such as those described in Remington: The Science and Practice of Pharmacy (Gennaro and Gennaro, eds, 20th edition, Lippincott Williams & Wilkins, 2000); Theory and Practice of Industrial Pharmacy (Lachman et al., eds., 3rd edition, Lippincott Williams & Wilkins, 1986); Encyclopedia of Pharmaceutical Technology (Swarbrick and Boylan, eds., 2nd edition, Marcel Dekker, 2002).

DAST and the pharmaceutical compositions of the present invention can be in any suitable form, without limitation. Forms suitable for oral use, include, but are not limited to, tablets, troches, lozenges, aqueous or oily suspensions, dispersible powders or granules, emulsions, hard or soft capsules, solutions, syrups and elixirs. Compositions intended for oral use may be prepared according to any method known to the art for the manufacture of pharmaceutical compositions.

DAST can be formulated with other ingredients, e.g., “pharmaceutically acceptable carriers” or “excipients” to indicate they are combined with the active drug and can be administered safely to a subject for therapeutic purposes. These include, but are not limited to, antioxidants, preservatives, dyes, tablet-coating compositions, plasticizers, inert carriers, excipients, polymers, coating materials, osmotic barriers, devices and agents which slow or retard solubility, etc.

Pharmaceutical compositions intended for oral use may be prepared according to any suitable method known to the art for the manufacture of pharmaceutical compositions. Such compositions may contain one or more agents selected from the group consisting of diluents, sweetening agents, flavoring agents, coloring agents and preserving agents in order to provide palatable preparations.

Non-toxic pharmaceutically acceptable excipients that are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; and binding agents, for example magnesium stearate, stearic acid or talc.

Pharmaceutical compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions containing the active materials in admixture with excipients suitable for the manufacture of aqueous suspensions may also be used. Such excipients are suspending agents, for example sodium carboxymethylcellulose, methylcellulose, hydroxypropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethylene oxycetanol, or condensation products of ethylene oxide with partial esters derived from fatty acids and hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example, sweetening, flavoring and coloring agents, may also be present.

DAST and the pharmaceutical compositions of the present invention may also be in the form of non-aqueous liquid formulations, e.g., oily suspensions which may be formulated by suspending the active ingredients in a vegetable oil, for example arachis oil, olive oil, sesame oil or peanut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

DAST and the pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

DAST and the pharmaceutical compositions of the invention may also be administered in the form of suppositories for rectal or vaginal administration of the drug. These compositions can be prepared by mixing the drug with a suitable non-irritating excipient which is solid at ordinary temperatures but liquid at the rectal temperature or vaginal temperature and will therefore melt in the rectum or vagina to release the drug. Such materials include cocoa butter and polyethylene glycols.

DAST and the pharmaceutical compositions of the invention may also be administrated transdermally using methods known to those skilled in the art (see, for example: Chien; “Transdermal Controlled Systemic Medications”; Marcel Dekker, Inc.; 1987. Lipp et al. WO94/04157). For example, a solution or suspension of a compound of Formula I in a suitable volatile solvent optionally containing penetration enhancing agents can be combined with additional additives known to those skilled in the art, such as matrix materials and bacteriocides. After sterilization, the resulting mixture can be formulated following known procedures into dosage forms. In addition, on treatment with emulsifying agents and water, a solution or suspension of a compound of Formula I may be formulated into a lotion or salve.

Suitable solvents for processing transdermal delivery systems are known to those skilled in the art, and include lower alcohols such as ethanol or isopropyl alcohol, lower ketones such as acetone, lower carboxylic acid esters such as ethyl acetate, polar ethers such as tetrahydrofuran, lower hydrocarbons such as hexane, cyclohexane or benzene, or halogenated hydrocarbons such as dichloromethane, chloroform, trichlorotrifluoroethane, or trichlorofluoroethane. Suitable solvents may also include mixtures of one or more materials selected from lower alcohols, lower ketones, lower carboxylic acid esters, polar ethers, lower hydrocarbons, halogenated hydrocarbons.

Suitable penetration enhancing materials for transdermal delivery system are known to those skilled in the art, and include, for example, monohydroxy or polyhydroxy alcohols such as ethanol, propylene glycol or benzyl alcohol, saturated or unsaturated C8-C18 fatty alcohols such as lauryl alcohol or cetyl alcohol, saturated or unsaturated C8-C18 fatty acids such as stearic acid, saturated or unsaturated fatty esters with up to 24 carbons such as methyl, ethyl, propyl, isopropyl, n-butyl, sec-butyl, isobutyl, tertbutyl or monoglycerin esters of acetic acid, capronic acid, lauric acid, myristinic acid, stearic acid, or palmitic acid, or diesters of saturated or unsaturated dicarboxylic acids with a total of up to 24 carbons such as diisopropyl adipate, diisobutyl adipate, diisopropyl sebacate, diisopropyl maleate, or diisopropyl fumarate. Additional penetration enhancing materials include phosphatidyl derivatives such as lecithin or cephalin, terpenes, amides, ketones, ureas and their derivatives, and ethers such as dimethyl isosorbid and diethyleneglycol monoethyl ether. Suitable penetration enhancing formulations may also include mixtures of one or more materials selected from monohydroxy or polyhydroxy alcohols, saturated or unsaturated C8-C18 fatty alcohols, saturated or unsaturated C8-C18 fatty acids, saturated or unsaturated fatty esters with up to 24 carbons, diesters of saturated or unsaturated discarboxylic acids with a total of up to 24 carbons, phosphatidyl derivatives, terpenes, amides, ketones, ureas and their derivatives, and ethers.

Suitable binding materials for transdermal delivery systems are known to those skilled in the art and include polyacrylates, silicones, polyurethanes, block polymers, styrenebutadiene copolymers, and natural and synthetic rubbers. Cellulose ethers, derivatized polyethylenes, and silicates may also be used as matrix components. Additional additives, such as viscous resins or oils may be added to increase the viscosity of the matrix.

Compositions comprising precursors can also be formulated for controlled release, where release of the active ingredient is regulated or modulated to achieve a desired rate of delivery into the systemic circulation. A controlled release formulation can be pulsed, delayed, extended, slow, steady, immediate, rapid, fast, etc. It can comprise one or more release formulations, e.g. extended- and immediate-release components. Extended delivery systems can be utilized to achieve a dosing internal of once every 24 hours, once every 12 hours, once every 8 hours, once every 6 hours, etc. The dosage form/delivery system can be a tablet or a capsule suited for extended release, but a sustained release liquid or suspension can also be used. A controlled release pharmaceutical formulation can be produced which maintains the release of, and or peak blood plasma levels of DAST.

In preferred solid oral pharmaceutical compositions according to the invention, at least 25% of DAST exists as a coprecipitate, more preferable at least 40% of DAST exists as a coprecipitate.

Micronization can be achieved by standard milling methods, preferably by air chat milling, known to a skilled person. The micronized form can have a mean particle size of from 0.5 to 10 μm, preferably from 1 to 6 μm, more preferably from 1 to 3 μm. The indicated particle size is the mean of the particle size distribution measured by laser diffraction known to a skilled person (measuring device: HELOS, Sympatec).

Pharmaceutical compositions which are preferred comprise DAST in a portion of at least 25%, preferably at least 45%, more preferably at least 50%, even more preferably at least 55%, by weight of the composition. Amounts of at least 62%, or at least 69%, or at least 75% by weight of the composition can be used under certain circumstances. Methods for preparing such formulations are disclosed in published international applications WO05/009961, published Feb. 3, 2005, and WO06/026500, published Mar. 9, 2006, which are incorporated herein by reference. The entire disclosure of all applications, patents and publications, cited above and in the figures are hereby incorporated by reference in their entirety.

Without further elaboration, it is believed that one skilled in the art, using the preceding description and information available in the art, can utilize the present invention to its fullest extent. One skilled in the art can easily ascertain the essential characteristics of this invention and, without departing from the spirit and scope thereof, make various changes and modifications of the invention to adapt it to various usages and conditions. For example, the preceding examples can be repeated with similar success by substituting the generically or specifically described reactants and/or operating conditions of this invention for those used in these examples. The preceding examples are, therefore, to be construed as merely illustrative, and not limitative of the remainder of the disclosure in any way whatsoever.

It should be apparent to one of ordinary skill in the art that changes and modifications can be made to this invention without departing from the spirit or scope of the invention as it is set forth herein.

In the foregoing and in the examples, all temperatures are set forth uncorrected in degrees Celsius and, all parts and percentages are by weight, unless otherwise indicated.

The topic headings set forth above are meant as guidance where certain information can be found in the application, but are not intended to be the only source in the application where information on such topic can be found.

1. A method of treating a cancer in a subject in need thereof, wherein said cancer has acquired resistance to a tyrosine kinase inhibitor, said method comprising:
administering to said subject, an effective amount of 4{4-[3-(4-chloro-3-trifluoromethylphenyl)-ureido]-3-fluorophenoxy}-pyridine-2-carboxylic acid methylamide of the formula I below including all polymorphs, hydrates, pharmaceutically acceptable salts, metabolites, prodrugs, solvates or combinations thereof.

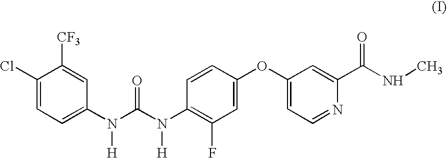

2. A method of treating a cancer in a subject in need thereof as in claim 1, wherein said cancer was initially sensitive to a tyrosine kinase inhibitor and acquired resistance to said tyrosine kinase inhibitor, said method comprising
administering to said subject, an effective amount of 4{4-[3-(4-chloro-3-trifluoromethylphenyl)-ureido]-3-fluorophenoxy}-pyridine-2-carboxylic acid methylamide of the formula I below including all polymorphs, hydrates, pharmaceutically acceptable salts, metabolites, prodrugs, solvates or combinations thereof.

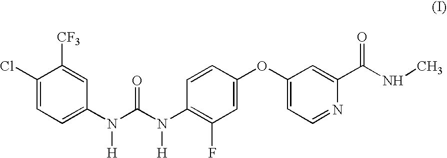

3. A method of claim 1, wherein said acquired resistance of said cancer is associated with a secondary mutation in a gene mutated in the primary tumor.

4. A method of claim 1, wherein one of the following kinase targets in the cancer cells acquired resistance to inhibition through gene mutation: PDGFR-alpha, PDGFR-beta, EGFR, VEGFR, VEGFR1, VEGFR2, VEGFR3, HER-2, KIT, FLT3, c-MET, FGFR, FGFR1, FGFR3, c-FMS, RET, ABL, ALK, ARG, NTRK1 m NTRK3, JAK2, or ROS.

5. A method of claim 1, wherein one of the following kinase targets in the cancer cells acquired resistance to inhibition through gene mutation at the kinase catalytic domain: BCR-ABL, KIT receptor, PDGF receptor, and EGF receptor.

6. A method as in claim 1 wherein the cancer has acquired resistance to one or more of the following tyrosine kinase inhibitors: AEE788, AMG 706, AMN107, ARRY-142886 (AZD6244), AZD2171, AZD0530, bevacizumab, BMS-354825, BMS-599626, CCI779, CEP-7055, cetuximab, CHIR-258, CI-1033, CP-724714, CP-547-632, erlotinib (tarceva or OSI774), gefitinib (Iressa), GW572016, GW786034, imatinib mesylate (STI57 or Gleevec), lapatinib ditosylate (GSK572016), PD 0173074, PD 0325901, PKC412, PTK787, rapamycin, sunitinib (sutent), SU5416, SU11248, SU6668, trastuzumab, XL647, ZD6474, and analogs and derivatives thereof.

7. A method as in claim 1 wherein the cancer has acquired resistance to one or more of the following tyrosine kinase inhibitors: 17-DMAG; 17-AAG; AG 9; AG 10; AG 1; AG 18; AG 30; AG 43; AG 82; AG 99; AG 112; AG 126; AG 183; AG 213; AG 370: AG 490; AG 494; AG 527; AG 537; AG 538; AG 555; AG 556; AG 592; AG 825; AG 835; AG 879; AG 957; AG 957; AG 1024; AG 1288; AG 1295; AG 1296; AG 1387; AG 1433; AG 1478; AGL 2043; AGL 2263; Aminogenistein; BPDQ; BPIQ-I; BPIQ-II; 4-[(3′-Bromo-4′-hydroxyphenyl)amino]-6,7-dimethoxyquinazoline (WHI-P154); 4-[(3-Bromophenyl)amino]-6,7-diethoxyquinazoline; Butein; (5-Butanoate-1H-2-indolyl) (1H-2-indolyl)-methanone; 4-[(4′-Chloro-2′-fluoro)phenylamino]-6,7-dimethoxyquinazoline; N-(4-Chlorophenyl)-2-[(pyridin-4-ylmethyl)amino]benzamide; CL-387785; Cucurbitacin I, Cucumis sativus L.; Curcumin, Curcuma longa L.; Daidzein; Damnacanthal; Daphnetin; 5′-Deoxy-5′-methylthioadenosine; 4-(3′,5′-Dibromo-4-hydroxyphenyl)amino-6,7-dimethoxyquinazoline (WHI-P97) ; (Z)-5-Bromo-3-(4,5,6,7-tetrahydro-1H-indol-2-ylmethylene)-1,3-dihydroindol-2-one; 2-(1,1-Dimethylethyl)-9-fluoro-3,6-dihydro-7H-benz[h]-imidaz[4,5-f]isoquinolin-7-one; 4-(6,7-Dimethoxy-4-quinazolinyl)-N-(4-phenoxyphenyl)-1-piperazinecarboxamide; 3-[(2,4-Dimethylpyrrol-5-yl)methylidene]-indolin-2-one (SU5416); (Z)-3-[(2,4-Dimethyl-3-(ethoxycarbonyl)pyrrol-5-yl)methylidenyl]indolin-2-one; DMBI; Emodin; Erbstatin Analog; Geldanamycin (Streptomyces hygroscopicus); Genistein; Genistin or GTP-14564.

8. A method as in claim 1 wherein the cancer has acquired resistance to one or more of the following tyrosine kinase inhibitors: Herbimycin A (Streptomyces sp.); 1,2,3,4,5,6-Hexabromocyclohexane; HNMPA-(AM)3; (5-Hydroxy-1H-2-indolyl) (1H-2-indolyl)-methanone; 4-(4′-Hydroxyphenyl)amino-6,7-dimethoxyquinazoline (WHI-P131); IGF-1R Inhibitor, PPP; I-OMe-AG 538; (Z,E)-3-(Imidazol-4-ylmethylene)indolin-2-one; [2-(1H-2-Indolylcarbonyl)-1H-5-indolyl]butanoate; Indirubin Derivative E804; K-252a, Nocardiopsis sp.; Lavendustin A; Lavendustin B; LFM-A11; LFM-A12; LFM-A13; MAZ51; 3-(1-Methyl-1H-indol-3-yl-methylene)-2-oxo-2,3-dihydro-1H-indole-5-sulfonamide; 2-Naphthyl-(N-isopropyl,N-benzyl)-b-aminoethylketone, HCl; 2-Naphthylvinyl Ketone; Oxindole I; PD 153035; PD 156273; PD 158780; PD 168393; PD 174265; Piceatannol; PP1 Analog; PP1 Analog II (1NM-PP1); PP2; PP3; Quercetin; Radicicol (Diheterospora chlamydosporia); RG-13022; 4-(4′-Phenoxyanilino)-6,7-dimethoxyquinazoline; p60v-src 137-157 Inhibitor Peptide (VAPSDSIQAEEWYFGKITRRE); ST638; SU11652; SU1498; SU4984; SU5402; SU5614; SU6656; (−)-Terreic Acid, Synthetic; 2′-Thioadenosine; Tyrene CR4; 3-(3-Thienyl)-6-(4-methoxyphenyl)pyrazolo[1,5-a]pyrimidine; ZM323881; ZM 39923; or ZM 449829.

9. A method as in claim 1 wherein the cancer has acquired resistance to gefitinib (Iressa).

10. A method as in claim 1 wherein the cancer has acquired resistance to imatinib.

11. A method as in claim 1 wherein the cancer has acquired resistance to tarceva.

12. A method as in claim 1 wherein the cancer has acquired resistance to erlotinib.

13. A method as in claim 1 wherein the cancer which is treated is:
CML (chronic myeloid leukemia),
ALL (acute lymphoblastic leukemia),
AML (acute myelogenous leukemia),
T-ALL (T-Cell acute lymphoblastic leukemia),
ALCL (acute lymphoblast cell leukemia),
EMS (8p11 myeloproliferative syndrome),
aCML (atypical chronic myelogenous leukemia),
MM (multiple myeloma),
T-lymphoma,
MDS (myelodysplastic (syndrome),
HES (hypereosinophilic syndrome),
SM (systemic mastocytosis), and
CMML (chronic myelomonocytic leukemia),
IMT (inflammatory myofibroblastic tumor),
NSCLC (non-small cell lung cancer),
glioblastoma, SCCHN (squamous cell carcinoma of the head and neck),
ovarian cancer,
RCC (renal cell carcinoma),
pancreatic cancer,
colorectal cancer,
breast cancer,
lung cancer,
seminoa,
sarcomas,
musculoskeletal tumors,
renal papillary carcinoma,
malignant melanoma,
PTC (papillary thyroid cancer),
congenital fibrosarcoma,
mesoblastic nephroma,
secretory breast carcinoma,
osteosarcoma,
PAIS (pulmonary artery intimal sarcoma),
DFSP (dermatofibrosarcoma protuberans),
FMTC (familial medullary thyroid carcinoma),
MEN-2B, radiation associated papillary thyroid cancer,
astrocytoma,
breast cancer,
prostate cancer or
renal cancer.

14. A method of claim 2 wherein the cancer which is treated is
Adenocarcinoma of the Colon;
Adenocarcinoma of the Esophagus;
Adenocarcinoma of the Lung;
Adenocarcinoma of the Prostate;
Adenocarcinoma of the Rectum;
Advanced Adult Primary Liver Cancer;
Advanced Non-Nasopharyngeal Head and Neck Carcinoma;
Anaplastic Astrocytoma;
Anaplastic Oligodendroglima;
Anaplastic Thyroid Cancer;
Bladder Cancer;
Brain Tumor;
Breast Cancer;
Breast Cancer in Situ;
Breast Neoplasms;
Bronchoalveolar Cell Lung Cancer;
Cancer of the Fallopian Tube;
Carcinoma,
Squamous Cell;
Cervix Neoplasms;
Colon Cancer;
Colorectal Cancer;
Epithelial Mesothelioma;
Esophageal Cancer;
Esophagogastric Cancer;
Follicular Thyroid Cancer;
Gastric Cancer;
Gastrinoma;
Gastrointestinal Carcinoid;
Giant Cell Glioblastoma; Glioblastoma;
Glioblastoma Multiforme;
Head and Neck Cancer;
Hepatocellular Carcinoma;
Hypopharyngeal Cancer;
Inoperable Locally Advanced Squamous Cell Carcinoma of Head and Neck;
Insulinoma;
Intraductal Breast Carcinoma;
Islet Cell Carcinoma;
Large Cell Lung Cancer;
Laryngeal Cancer;
Lip and Oral Cavity Cancer;
Lip Cancer;
Liver Cancer;
Lung Adenocarcinoma With Bronchiolo-Alveolar Feature;
Lung Cancer;
Male Breast Cancer;
Medullary Thyrod Cancer;
Meningeal Tumors;
Metastatic Colorectal Cancer;
Metastatic Gastrointestinal Carcinoid Tumor;
Metastatic Pancreatic Carcinoma;
Mixed Gliomas;
Myelogenous Leukemia,
Acute;
Nasopharyngeal Carcinoma;
Neuroblastoma;
Non-Metastatic (T2-T4, N0-N3, M0; Stages II and III) and Histologically-Confirmed Intestinal GC;
Non-Metastatic Prostate Cancer;
Nonresectable Adrenocortical Carcinoma;
Non-Small Cell Lung Cancer;
Nose Cancer;
Oligodendroglial Tumors;
Oral Cancer;
Oropharyngeal Cancer;
Osteosarcoma; Ovarian Cancer;
Ovarian Neoplasms;
Pancreatic Cancer;
Papillary Thyroid Cancer;
Peritoneal Carcinoma;
Pharynx Cancer;
Pneumonic-Type Adenocarcinoma (P-ADC);
Primary Hepatocellular Carcinoma;
Prostate Cancer;
Rectal Cancer;
Recurrent Adult Primary Liver Cancer;
Recurrent Breast Cancer;
Recurrent Colon Cancer;
Recurrent Endometrial Cancer;
Recurrent Esophageal Cancer;
Recurrent Glioblastoma;
Recurrent Rectal Cancer;
Recurrent Skin Cancer;
Refractory Germ Cell Tumors Expressing EGRF;
Renal Cell Cancer;
Rhabdomyosarcomas;
Sarcomatous Mesothelioma;
Skin Cancer;
Soft Tissue Sarcoma;
Squamous Cell Carcinoma of the Esophagus;
Squamous Cell Carcinoma of the Head and Neck;
Squamous Cell Carcinoma of the Skin;
Squamous Cell Lung Cancer;
Stage II Esophageal Cancer;
Stage III Esophageal Cancer;
Synovial Sarcoma;
Thorax and Respiratory Cancer;
Throat Cancer; Thyroid Cancer;
Transitional Cell Cancer of the Renal Pelvis and Ureter;
Transitional Cell Carcinoma of the Bladder;
Tubal Carcinoma;
Unspecified Childhood Solid Tumor;
Untreated Childhood Brain Stem Glioma or
Urethral Cancer.

15. A method of claim 2 wherein the cancer which is treated is
Adenocarcinoma;
Adenocarcinoma of the Colon;
Adenocarcinoma of the Esophagus;
Adenocarcinoma of the Lung;
Adenocarcinoma of the Pancreas;
Adenocarcinoma of the Prostate;
Adenocarcinoma of the Stomach;
denosquamous Cell Lung Cancer;
Adult Giant Cell Glioblastoma;
Advanced Adult Primary Liver Cancer;
Advanced NSCLC;
Advanced Solid Tumors;
Anaplastic Astrocytoma;
Anaplastic Olligodendroglioma;
Andrigen Deprivation Therapy;
Bladder Cancer;
Brenner Tumor;
Bronchoalveolar
Cell Lung Cancer;
Childhood Brain Tumor;
Childhood Cerebellar Astrocytoma;
Childhood Cerebral Astrocytoma;
Childhood Ependymoma;
Childhood Malignant; Germ Cell Tumor;
Childhood Oligodendroglioma;
Colorectal Cancer;
ECOG;
Endometrial Adenocarcinoma;
Endometrial Adenosquamous Cell;
Esophageal Cancer;
Extrahepatic Bile Duct Cancer;
Fallopian Tube Cancer;
Fallopian Tube Cancer;
Female Reproductive Cancer;
Gallbladder Cancer;
Gastric Cancer;
Gastrointestinal Cancer;
Glioblastoma Multiforme;
Gliosarcoma;
Head and Neck Cancer;
Head and Neck Neoplasms;
High-Grade Childhood;
Cerebral Astrocytoma;
Hormone Sensitive Metastatic Breast Cancer;
Hypopharyngeal Cancer;
Kidney and Urinary Cancer;
Laryngeal Cancer; Localized Unresectable Adult Primary liver Cancer;
Low-Grade Childhood Cerebral Astrocytoma;
Lung Adenocarcinoma With Bronchiolo-Alveolar Feature;
Male Breast Cancer;
Meningioma; Mesothelioma;
Mixed Gliomas;
Nasopharyngeal Cancer;
Neoplasms;
Neurofibrosarcoma;
Non-Metastatic Prostate Cancer;
Non-Small-Cell Lung;
Oral Cavity Cancer;
Oropharyngeal Cancer;
Ovarian Cancer;
Ovarian Epithelial Cancer;
Ovarian Neoplasms;
Pancreatic Cancer;
Peritoneal Cavity Cancer;
Pharynx Cancer;
Pharynx Neoplasms;
Pneumonic-Type Adenocarcinoma (P-ADC);
Primary Hepatocellular Carcinoma;
Primary Liver Cancer;
Prostate Cancer;
Androgen Independent;
Pulmonary Diseases;
Recurrent Adult Brain Tumor;
Recurrent Adult Primary Liver Cancer;
Recurrent Breast Cancer;
Recurrent Cervical Cancer;
Recurrent Endometrial Cancer;
Recurrent Esophageal Cancer;
Recurrent Pancreatic Cancer
Recurrent Renal Cell Cancer;
Relapsed/Refractory Non-Small-Cell Lung Cancer;
Renal Cell Carcinoma; Rising Prostate Specific Antigen (PSA);
Soft Tissue Sarcoma; Squamous Cell Carcinoma;
Squamous Cell Carcinoma of the Esophagus;
Squamous Cell Carcinoma of the Lip and Oral Cavity;
Squamous Cell Carcinoma of the Oropharynx;
Stage II Pancreatic Cancer; Stage III Pancreatic Cancer;
Stage IIIA Non-Small Cell Lung Cancer;
Stage IIIB or IV Non-Small Cell Lung Cancer;
Stage IV Breast Cancer; Stage IV Colon Cancer;
Stage IV Endometrial Cancer; Stage IV Rectal Cancer;
Thorax and Respiratory Cancer;
Transitional Cell Carcinoma of the Bladder;
Tumors Metastatic to Brain;
Unspecified Adult Solid Tumor; or
Upper Aerodigestive Tract Neoplasms.

16. A method of treating a cancer in a subject in need thereof said cancer having a primary and/or secondary gene mutation associated with resistance or acquired resistance to tyrosine kinase inhibitors, said method comprising:
administering to said subject, an effective amount of 4{4-[3-(4-chloro-3-trifluoromethylphenyl)-ureido]-3-fluorophenoxy}-pyridine-2-carboxylic acid methylamide of the formula I below including all polymorphs, hydrates, pharmaceutically acceptable salts, metabolites, ester prodrugs, solvates or combinations thereof.

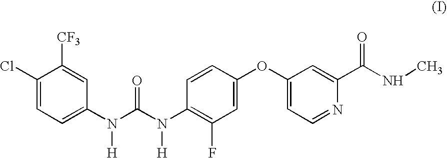

17. A method of treating a cancer in a subject in need thereof, comprising:
administering an effective amount of DAST to said subject having a cancer, wherein said cancer has acquired resistance to a tyrosine kinase inhibitor.
